(19) United States
(12) Patent Application Publication
(10) Pub. No.: US 2010/0200435 A1
(43) Pub. Date: Aug. 12, 2010

(54) DISPOSABLE TOOTHBRUSH

(76) Inventor: Henry BAUS, Medellin (CO)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/368,471

(22) Filed: Feb. 10, 2009

(51) Int. Cl. A61B 19/02; A61C 15/04

(52) U.S. Cl. 206/63.5; 132/321

(57) ABSTRACT

A tooth care kit, comprising a set of disposable handle attachments and a set of disposable brush attachments each arranged to interfit with a corresponding one of the set of disposable handle attachments.

### BACKGROUND

Practicing frequent oral hygiene is beneficial to human health. A user brushing their teeth and/or gums alone or in combination with flossing increases the health of the user. For example, tooth illnesses may be minimized and/or prevented by such oral hygiene by controlling fungal and bacterial growth.

### DESCRIPTION OF THE DRAWINGS

One or more embodiments are illustrated by way of example, and not by limitation, in the figures of the accompanying drawings, wherein elements having the same reference numeral designations represent like elements throughout and wherein:

FIG. 1 is a perspective view of components of a toothbrush in a disassembled state according to an embodiment;

FIGS. 2A-C are a sequence of connecting/disconnecting a head segment and a base segment to form a toothbrush according to an embodiment;

FIG. 3 is a perspective view of a toothbrush in an assembled state according to an embodiment;

FIG. 4 is a perspective view of components of a toothbrush in a disassembled state according to an embodiment;

FIG. 5 is a perspective view of a toothbrush in a storage state according to an embodiment;

FIGS. 6A-B are side sectional views of a toothbrush according to an embodiment in a storage state and in an assembled state, respectively;

FIG. 7 is a perspective view of a tooth care kit according to an embodiment;

FIG. 8 is a top perspective view of a tooth care kit according to an embodiment;

FIG. 9 is a perspective view of an example distribution package according to an embodiment including a tooth care kit;

FIG. 10 is a toothbrush according to another embodiment in a disassembled state; and

FIGS. 11A-C are a sequence of connecting/disconnecting a head segment and a base segment to form a toothbrush according to another embodiment.

### DETAILED DESCRIPTION

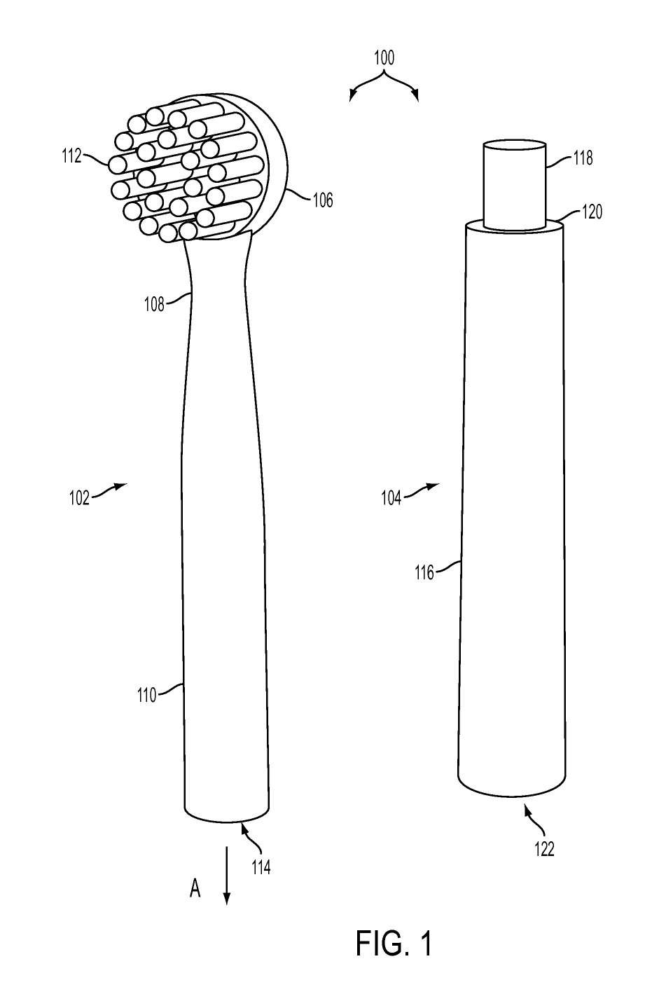

FIG. 1 depicts a perspective view of a disposable toothbrush 100 in a disassembled state according to an embodiment. Disposable toothbrush 100 comprises an upper portion 102 (also referred to as a head segment) and a lower portion 104 (also referred to as a base segment). Head segment 102 placed in a cooperatively coupled arrangement with base segment 104 comprises toothbrush 100. In at least some embodiments, head segment 102 retains base segment 104 by an interfit compression arrangement. In at least some other embodiments, head segment 102 retains base segment 104 by a threaded arrangement.

Head segment 102 comprises a brush head 106 coupled, via a neck portion 108, to an upper join portion 110. In at least one embodiment, head segment 102 is 69 millimeters (mm) in length. Brush head 106 comprises a plurality of bristle groups 112 connected to a face of the brush head. In at least some embodiments, bristle groups 112 extend perpendicularly from the face of brush head 106. In at least some embodiments, bristle groups 112 extend from the same face of brush head 106. In at least some embodiments, each bristle group 112 comprises individual bristles having a similar length whereas in other embodiments, different length bristles and/or bristle groups 112 may be connected with brush head 106.

In at least one embodiment, brush head 106 comprises twenty-eight (28) groups of bristles. In at least one embodiment, each bristle group 112 comprises 52 NYLON micro bristles, e.g., as manufactured by DUPONT, or biodegradable bristles from animal or plant-based material.

Brush head 106, in at least some embodiments, is cylindrically-shaped with bristle groups 112 extending from one face of the cylinder. Brush head 106 connects with neck portion 108 at a side and with the neck portion extending radially from the brush head. In at least some embodiments, brush head 106 may comprise a different shape and/or size configuration, e.g., parallelepiped, elliptical, trapezoidal, cylindrical, etc.

Neck portion 108 connects upper join portion 110 with brush head 106. Neck portion 108 comprises a middle portion between upper join portion 110 and brush head 106 which tapers to a narrower diameter in comparison with the upper join portion 110. In at least some embodiments, neck portion 108 connects brush head 106 and upper join portion 110 directly without use of a tapered portion. In at least some embodiments, neck portion 108 is incorporated as a part of upper join portion 110 with or without a tapered portion.

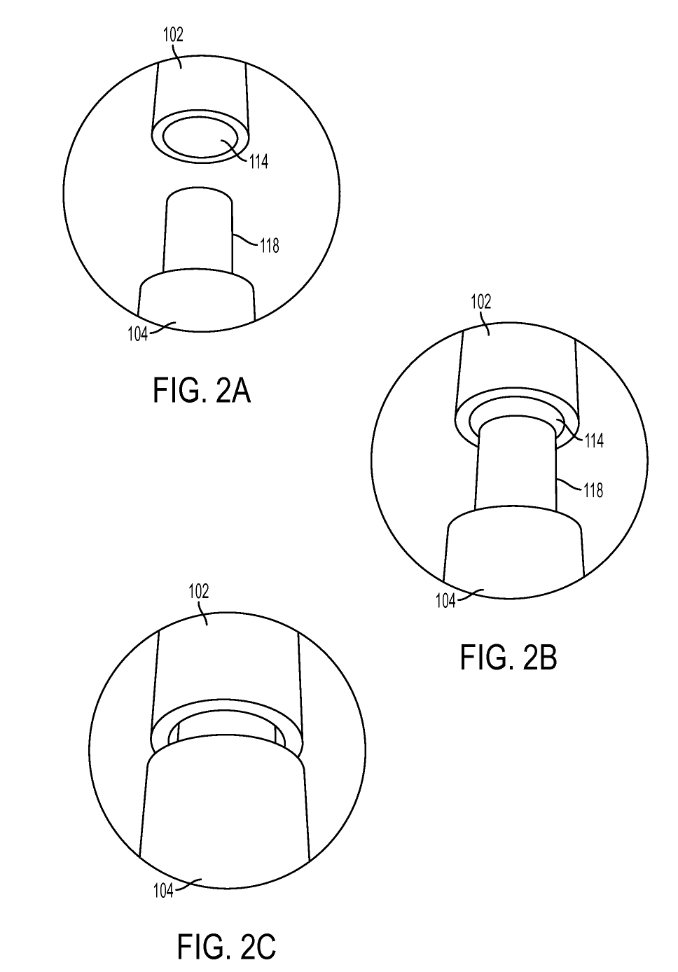

Upper join portion 110 is an elongated cylindrical segment which extends longitudinally along a radial axis A from brush head 106. Upper join portion 110 connects with neck portion 108 at an end proximate brush head 106. Upper join portion 110 is arranged to connect with base segment 104 at a distal end from neck portion 108. The distal end of upper join portion 110 comprises a receptacle 114 configured to receive at least a portion of base segment 104 to form toothbrush 100. In at least some embodiments, receptacle 114 is a cylindrical depression extending into upper join portion 110. Connection of head segment 102 and base segment 104 is discussed more fully with respect to FIG. 2.

In at least some embodiments, upper join portion 110 extends longitudinally along radial axis A without varying diameter. In at least some embodiments, upper join portion 110 extends longitudinally along radial axis A increasing in diameter from the end proximate neck portion 108 toward the opposite end.

In at least some embodiments, upper join portion 110 is sized in outer diameter sufficient to fit inside at least a portion of base segment 104.

In at least one embodiment, receptacle 114 extends longitudinally inward of upper join portion 110 to form a volume for retaining an amount of dental cleaning product, e.g., toothpaste, for a single use of toothbrush 100. A removable cover, e.g., paper-based or plastic-based, covers receptacle 114 at the end of upper join portion 110 retaining the toothpaste inside the upper join portion.

Base segment 104 comprises a generally elongated cylindrical shape configured to connect with head segment 102 to form toothbrush 100. In at least one embodiment, base segment 104 is 51 mm long. Base segment 104 comprises a grip segment 116 and a protruding connection member 118 located at one end of the grip segment. Grip segment 116 is an elongated cylindrical shape generally tapering in diameter from one end to the end comprising connection member 118. In at least some embodiments, grip segment 116 does not taper or change diameter from one end to the other.

Connection member 118 is an elongated cylindrical shape formed at one end of grip segment 116. A ledge 120 is formed at the connection of connection member 118 and group segment 116, i.e., the diameter of base segment 104 reduces at the transition from grip segment 116 to connection member 118. Connection member 118 is sized in diameter and height sufficient to interfit with upper join portion 110 of head segment 102, i.e., in at least some embodiments specifically connection member 118 is sized to interfit with receptacle 114.

Grip segment 116 also comprises a base receptacle 122 sized to receive at least a portion of head segment 102 within base segment 104. In at least some embodiments, base receptacle 122 is a cylindrical depression extending into base segment 104.

In at least one embodiment, head segment 102 and base segment 104 comprise biodegradable plant-based material such as wood or bamboo. In at least some embodiments, at least upper join portion 110, neck portion 108, and brush head 106 comprise a biodegradable plant-based material.

In at least one embodiment, toothbrush 100 is directed to a one-time and/or one-day use by a user and is intended to be disposed after such use.

FIGS. 2A-C depict a sequence of connecting/disconnecting head segment 102 and segment 104 to form toothbrush 100. Beginning at FIG. 2A, head segment 102 is axially aligned with base segment 104 such that receptacle 114 is positioned adjacent connection member 118. As head segment 102 and base segment 104 are axially moved toward each other, connection member 118 aligns with receptacle 114 as depicted in FIG. 2B. Further movement of head segment 102 and base segment 104 toward each other results in the insertion of connection member 118 into receptacle 114 just prior to complete formation of toothbrush 100 as depicted in FIG. 2C.

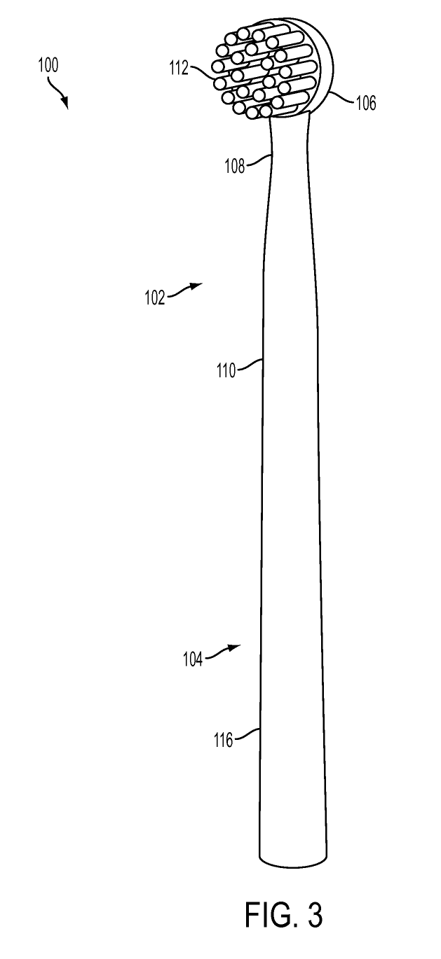

FIG. 3 depicts a perspective view of assembled toothbrush 100. In at least one embodiment, assembled toothbrush 100 is 120 mm in length. In use, a user may grip at least a part of base segment 104 and insert brush head 106 into the user's mouth in accordance with dental hygiene practices. In at least some embodiments, bristle groups 112 may comprise a toothpaste, gel, or other dental cleaning product for use in accordance with dental hygiene practices. In at least some other embodiments, a user may apply such cleaning product to the bristle groups 112.

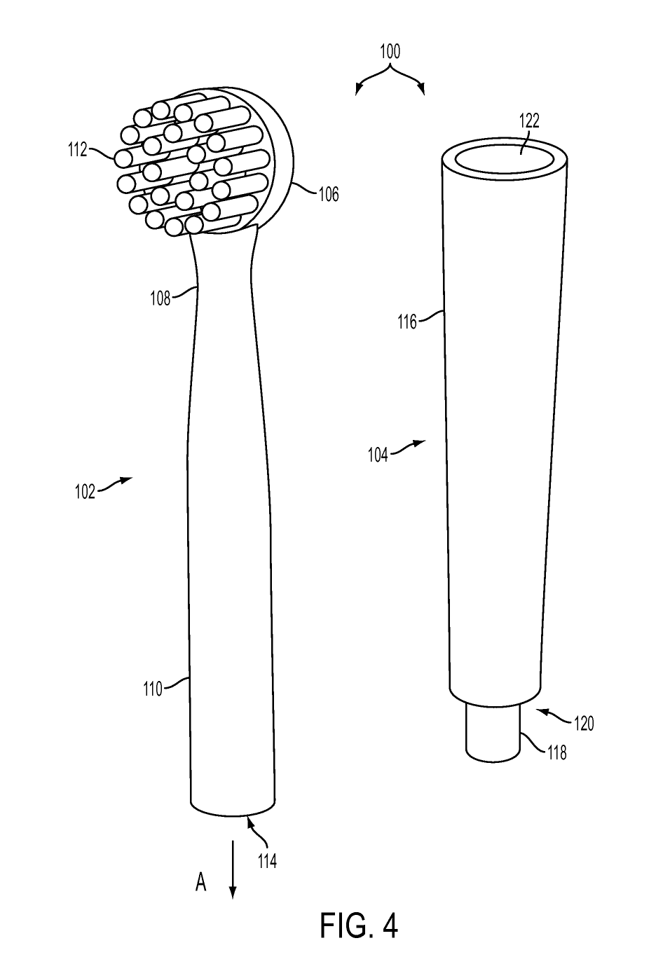

FIG. 4 depicts a perspective view of components of toothbrush 100 in a disassembled state according to an embodiment. In particular, FIG. 4 depicts a segment 104 in a reverse or upside down position with respect to FIG. 1 and depicting base receptacle 122.

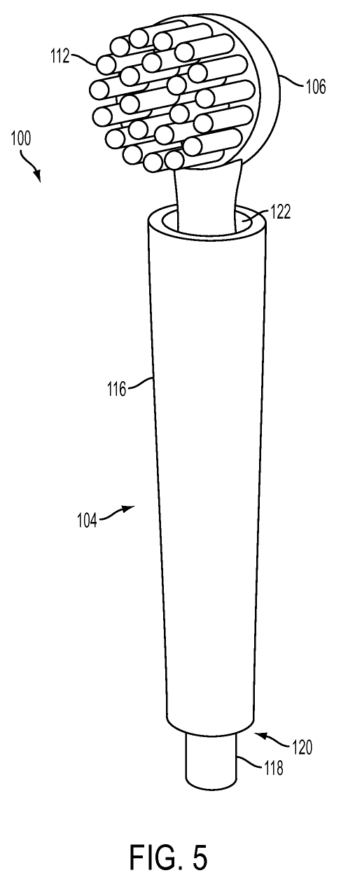

FIG. 5 depicts a perspective view of toothbrush 100 in a storage state according to an embodiment. In the storage state, toothbrush 100 and the components thereof require less volume to store with respect to disassembled state (FIG. 1) or assembled state (FIG. 3). Further, in the storage state toothbrush 100 is shorter in overall length than in assembled state, e.g., FIG. 3. Head segment 102 is positioned within base segment 104 in the storage state, e.g., upper join portion 110 is positioned within a portion of grip segment 116. As depicted, upper join portion 110 slides inside base receptacle 122 with brush head 106 extending outside the base receptacle.

In at least some embodiments, more or less of head segment 102 length extends outside base segment 104.

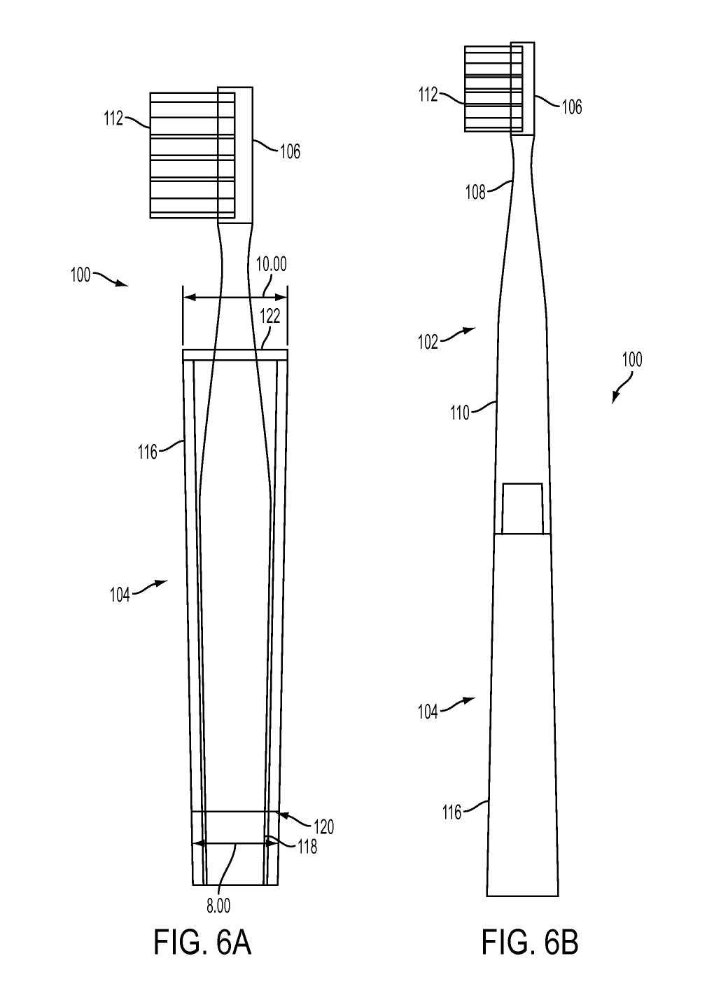

FIGS. 6A-B depict side sectional views of toothbrush 100 in a storage state and in an assembled state, respectively. FIG. 6A depicts a side cutaway section view of toothbrush 100 in a storage state. FIG. 6B depicts a side cutaway section view of toothbrush 100 in an assembled state.

As depicted in FIG. 6A, base receptacle 122 is sufficiently deep to receive inside a majority of the length of upper join portion 110 of head segment 102. In at least some embodiments, base receptacle 122 may be of a depth sufficient to receive at least fifty percent (50%) of upper join portion 110. In at least some embodiments, base receptacle 122 may be of a depth sufficient to receive at least seventy-five percent (75%) of upper join portion 110.

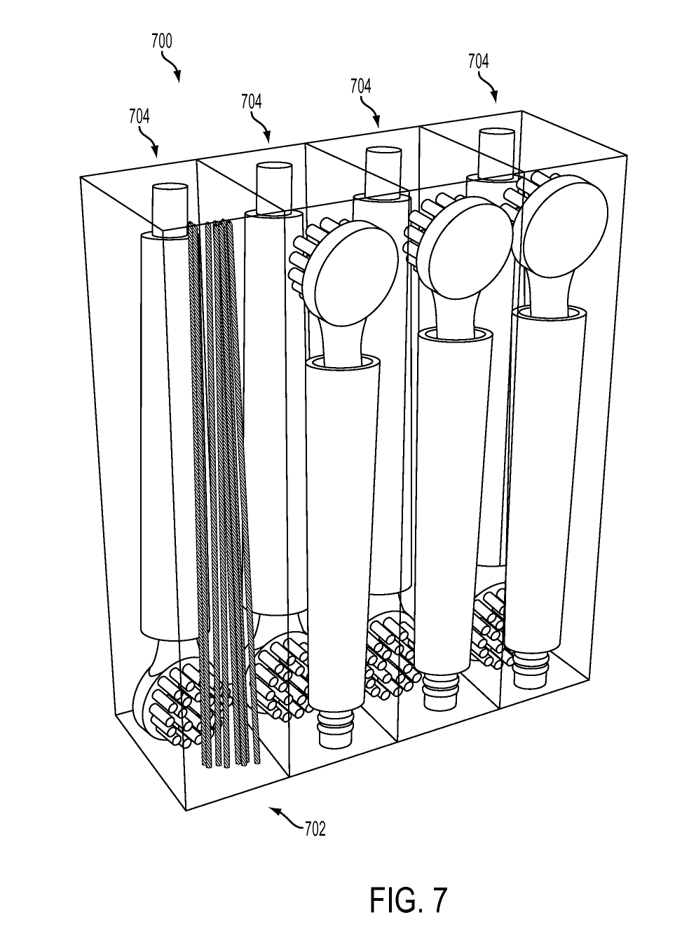

FIG. 7 depicts a perspective view of a tooth care kit 700 according to an embodiment which comprises a set of seven (7) toothbrushes 100, each in a storage state, and a set of floss threads 702. In at least some other embodiments, greater or fewer number of toothbrushes 100 may be packaged together in a tooth care kit. FIG. 7 depicts a tooth care kit 700 in which the packaging comprises transparent plastic. In at least some embodiments, tooth care kit 700 may comprise a different material and/or opaque or less transparent material.

Tooth care kit 700 comprises a set of seven (7) floss threads in correspondence with the number of toothbrushes in the kit. In at least some embodiments, greater or fewer number of floss threads may be packaged in a particular tooth care kit. In at least some embodiments, there is a one-to-one correspondence between the number of floss threads and the number of toothbrushes. In at least some other embodiments, there is no predetermined relationship between the number of floss threads and the number of toothbrushes. In at least one embodiment, the floss threads are 0.45 centimeters (cm) in length.

Tooth care kit 700 is a parallelepiped or box-like structure divided into four (4) receiving slots 704 arranged to receive the seven toothbrushes 100 and the floss threads 702 in corresponding slots. Each slot 704 receives at least one toothbrush 100 and either one other toothbrush or the set of floss threads 702 therein. In at least one embodiment, tooth care kit 700 is 8 cm in height, 5.6 cm in length, and 2.3 cm in width. In at least some embodiments, tooth care kit 700 is sized to fit into a man or woman's clothing pocket, purse, or bag to enable ease of transport.

Tooth care kit 700 stores seven toothbrushes 100 where each toothbrush corresponds to a single day use for a weeklong period of oral hygiene. In at least some embodiments, each toothbrush 100 comprises a day of the week identifier, e.g., an imprinted or affixed name of the day of the week, to indicate which toothbrush corresponds to the current day to minimize user confusion and minimize possible reuse of a used toothbrush. In at least some embodiments, each toothbrush 100 is colored a different color corresponding to a particular day of the week. For example, in accordance with a particular embodiment, Sunday is yellow, Monday is red, Tuesday is green, Wednesday is blue, Thursday is purple, Friday is orange, and Saturday is white.

Each pair of toothbrushes 100 inserted in a particular slot 704 are inserted upside down with respect to each other, i.e., head segment 102 of a first toothbrush is placed in proximate the upper join portion 110 of the head segment of a second toothbrush in the same slot. Positioning the paired toothbrushes in this manner further reduces the overall volume occupied by tooth care kit 700.

In at least some embodiments, tooth care kit 700 further comprises a lid or covering flap to secure the toothbrushes 100 and floss threads 702 within the container.

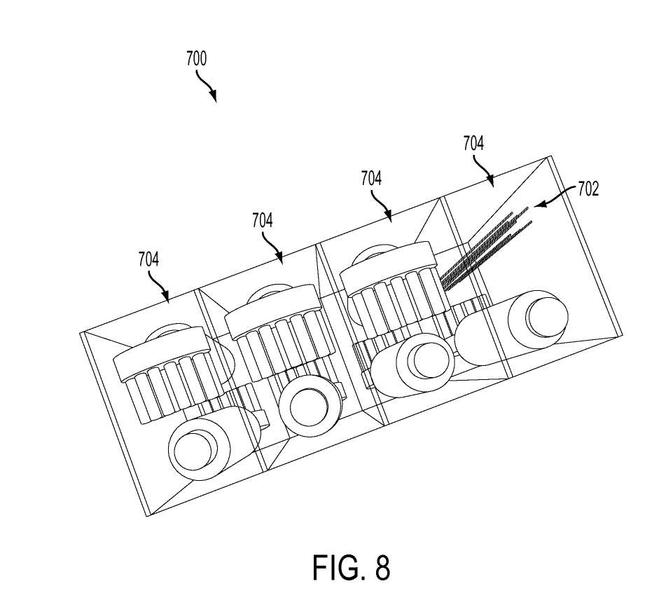

FIG. 8 depicts a top perspective view of tooth care kit 700 according to an embodiment.

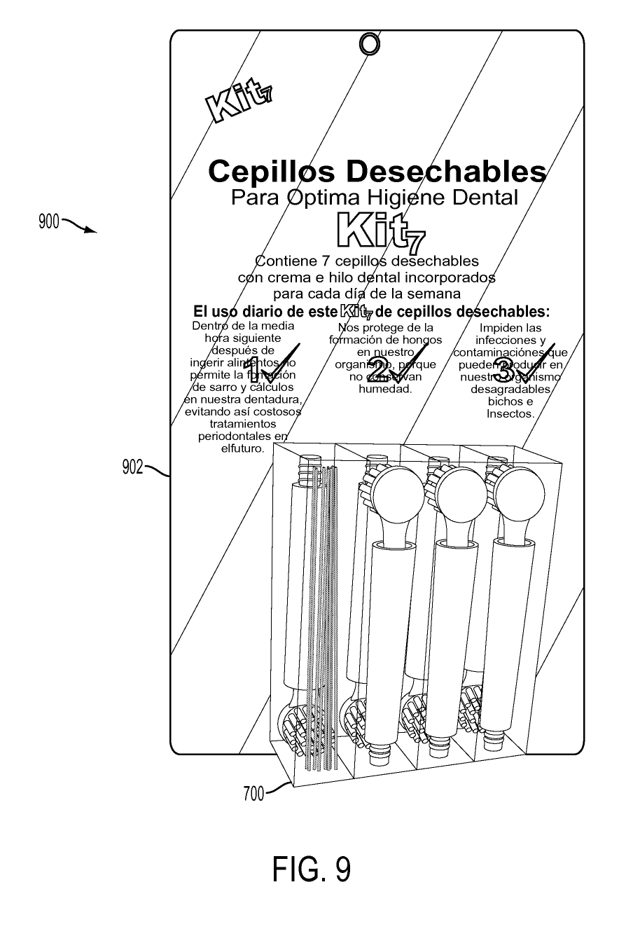

FIG. 9 depicts a perspective view of an example distribution package 900 of tooth care kit 700 according to an embodiment. Distribution package 900 comprises a display card 902, e.g., a rectangular-shaped backing card comprised of plastic, paper, or a combination thereof, to which is attached a tooth care kit 700 for display, e.g., in a retail establishment. Instructions for use of tooth care kit 700 may be found on a face of the backing card 902.

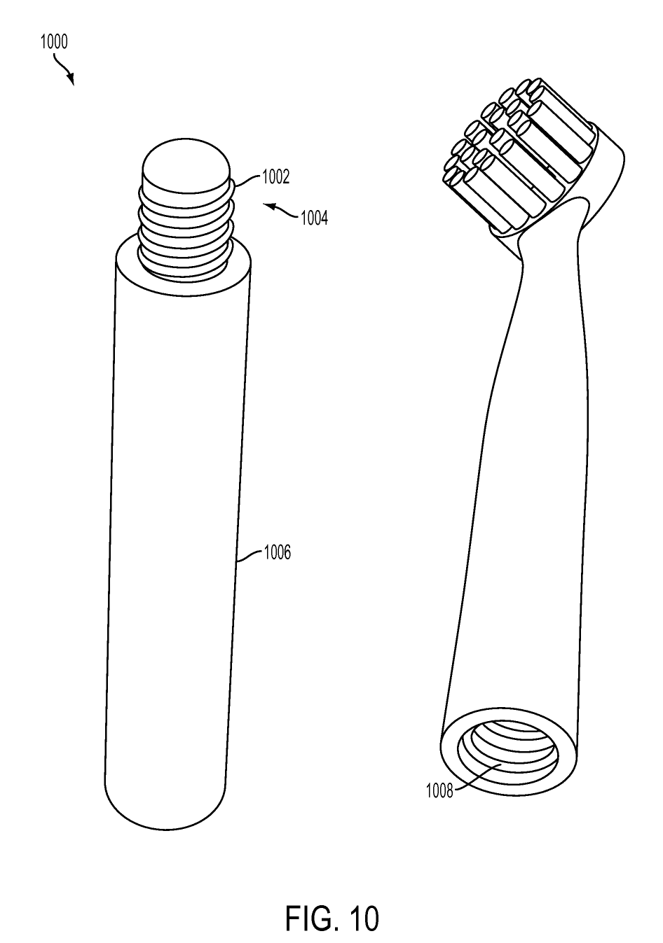

FIG. 10 depicts a toothbrush 1000 according to another embodiment in a disassembled state. Toothbrush 1000 is similar to toothbrush 100 (FIG. 1). However, toothbrush 1000 further comprises a threaded portion 1002 along a protruding connection member 1004 (similar to protruding connection member 118 (FIG. 1)) extending from grip segment 1006 (similar to grip segment 116 (FIG. 1)). Also, toothbrush 1000 comprises a corresponding inner receiving threaded portion 1008 extending inward of receptacle 1010 (similar to receptacle 114 (FIG. 1)) to the interior of upper join portion 1012 (similar to upper join portion 110 (FIG. 1)).

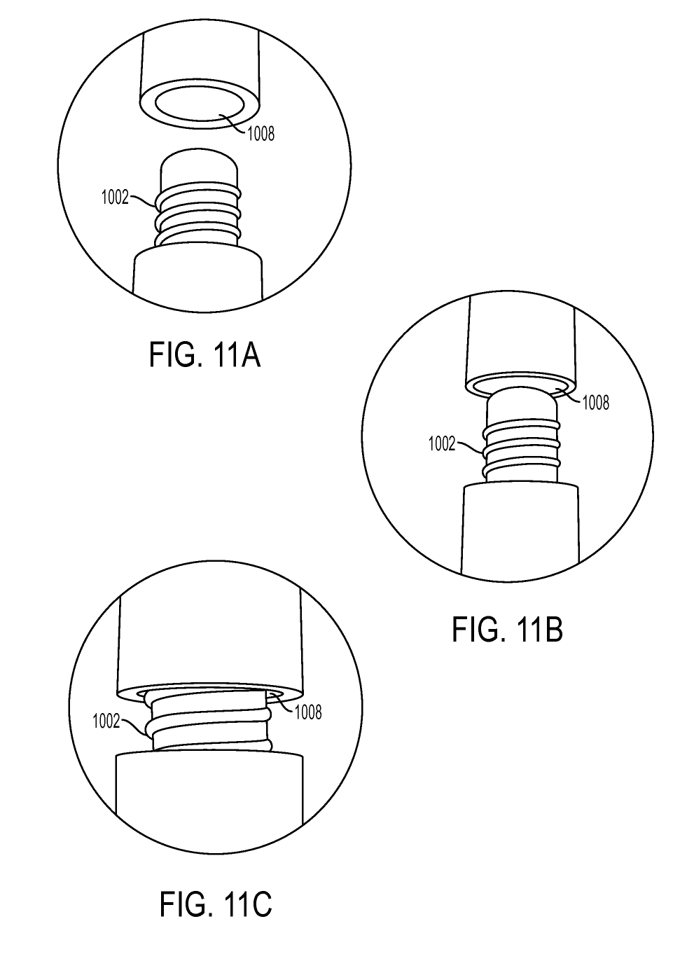

FIGS. 11A-C depict a sequence of connecting/disconnecting head segment 102 and segment 104 to form toothbrush 100. Beginning at FIG. 11A, head segment 102 is axially aligned with base segment 104 such that receptacle 114 is positioned adjacent connection member 118. As head segment 102 and base segment 104 are axially moved toward each other, connection member 118 aligns with receptacle 114 as depicted in FIG. 11B. Further movement of head segment 102 and base segment 104 toward each other and in a radial rotation results in the insertion of connection member 118 into receptacle 114 just prior to complete formation of toothbrush 100 as depicted in FIG. 11C. FIG. 11C depicts the threading of threaded portion 1002 into receiving threaded portion 1008.

It will be readily seen by one of ordinary skill in the art that the disclosed embodiments fulfill one or more of the advantages set forth above. After reading the foregoing specification, one of ordinary skill will be able to affect various changes, substitutions of equivalents and various other embodiments as broadly disclosed herein. It is therefore intended that the protection granted hereon be limited only by the definition contained in the appended claims and equivalents thereof.

1. A tooth care kit, comprising:
a set of disposable handle attachments; and
a set of disposable brush attachments each arranged to interfit with a corresponding one of the set of disposable handle attachments,
wherein at least one of the set of disposable brush attachments or the set of disposable handle attachments comprise biodegradable plant-based material.

2. The tooth care kit as claimed in claim 1, wherein each of the set of disposable handle attachments is retained interfit with the corresponding one of the set of disposable brush attachment using friction.

3. The tooth care kit as claimed in claim 1, wherein each of the set of disposable handle attachments comprises a first threaded end; and
each of the set of disposable brush attachments comprises a second threaded end arranged to interfit with the first threaded end.

4. The tooth care kit as claimed in claim 1, further comprising:
a case defining an interior volume and arranged to receive the set of disposable handle attachments and the set of disposable brush attachments within the interior volume.

5. The tooth care kit as claimed in claim 4, wherein the case comprises at least four slots arranged to each receive at least one of the set of disposable handle attachments and at least one of the set of disposable brush attachments.

6. The tooth care kit as claimed in claim 5, wherein each slot is arranged to receive at least two of the set of disposable handle attachments and at least one of the set of disposable brush attachments.

7. The tooth care kit as claimed in claim 1, further comprising a set of floss threads in relation to the number of disposable brush attachments.

8. The tooth care kit as claimed in claim 1, wherein the set of disposable brush attachments comprises seven disposable brush attachments.

9. The tooth care kit as claimed in claim 1, wherein at least one of the set of disposable brush attachments defines an interior volume having one end open to the exterior of the brush attachment.

10. The tooth care kit as claimed in claim 9, wherein the interior volume comprises
a dental cleaning product.

11. The tooth care kit as claimed in claim 9, wherein the at least one of the set of disposable brush attachments comprises a removably affixed cover positioned over the open end of the interior volume.

12. The tooth care kit as claimed in claim 1, wherein at least one of the set of disposable handle attachments or the set of disposable brush attachments further comprise a day of the week indication thereon.

13. A tooth care package, comprising:
a display card; and
a case attached to the display card, comprising:
a set of disposable handle attachments; and
a set of disposable brush attachments each arranged to interfit with a corresponding one of the set of disposable handle attachments,
wherein at least one of the set of disposable brush attachments or the set of disposable handle attachments comprise biodegradable plant-based material.

14. The tooth care kit as claimed in claim 1, wherein at least one of the set of disposable handle attachments defines an interior volume having one end open to the exterior of the handle attachment and sized to receive at least one of the set of disposable brush attachments at least partially within the interior volume.

15. The tooth care kit as claimed in claim 14, wherein a brush head of an at least partially received disposable brush attachment extends exterior to the interior volume.

16. The tooth care kit as claimed in claim 13, wherein at least one of the set of disposable handle attachments defines an interior volume having one end open to the exterior of the handle attachment and sized to receive at least one of the set of disposable brush attachments at least partially within the interior volume.

17. The tooth care kit as claimed in claim 16, wherein a brush head of an at least partially received disposable brush attachment extends exterior to the interior volume.
